(19) United States
(12) Patent Application Publication
(10) Pub. No.: US 2022/0280573 A1
(43) Pub. Date: Sep. 8, 2022

(54) HUMAN UMBILICAL CORD-DERIVED COMPOSITIONS AND USES THEREOF FOR TREATING NEUROPATHY

(71) Applicant: Axogen Corporation, Alachua, FL (US)

(72) Inventors: Kiri K. HAMAKER, Gainesville, FL (US); Tracy Love, Alachua, FL (US); Alec Simon, Alachua, FL (US)

(73) Assignee: Axogen Corporation, Alachua, FL (US)

(21) Appl. No.: 17/461,830

(22) Filed: Aug. 30, 2021

Related U.S. Application Data
(60) Provisional application No. 63/156,123, filed on Mar. 3, 2021.
(60) Provisional application No. 63/237,602, filed on Aug. 27, 2021.

(51) Int. Cl. A61K 35/51; A61K 45/06; A61K 9/06

(52) U.S. Cl. CPC A61K 35/51; A61K 45/06; A61K 9/06

(57) ABSTRACT

The present disclosure provides improved biomaterials extracted from human umbilical cord (hUC) material. The materials are mechanically disrupted to produce micronized particles and are further treated with a protease and optionally mixed with a gel-forming agent. The materials may have improved inflammatory/anti-inflammatory profiles and may provide particular utility in the treatment of peripheral neuropathy by local administration of the hUC extracts.

### CROSS-REFERENCE TO RELATED APPLICATIONS

This patent application claims the benefit of priority to U.S. Provisional Patent Application No. 63/156,123, filed on Mar. 3, 2021, and to U.S. Provisional Patent Application No. 63/237,602, filed on Aug. 27, 2021, the entireties of each of which are incorporated herein by reference.

### BACKGROUND

### 1. Technical Field

The present disclosure relates generally to the field of neurobiology, medicine, and medical procedures. More particularly, it concerns improved biomaterials extracted from human umbilical cord (hUC) material that have improved inflammatory/anti-inflammatory profiles and their use in the treatment of neuropathy.

### 2. Background

Painful neuropathy is largely treated non-surgically, often with systemic administration of analgesics or NSAIDs to treat pain. As a first line of defense, analgesics and NSAIDs may resolve neuropathic pain depending on the type of injury; however, if pain does not resolve with analgesic or NSAID treatment, secondary treatment options become more invasive or risky such as treatment with anti-inflammatory steroid medication (e.g., corticosteroids), anti-convulsant medication (e.g., pregabalin), or surgery become options.

Unfortunately, the limitations of current first line treatments such as analgesics and NSAIDs is that they are not effective in resolving painful neuropathy for many patients. Many patients will undergo secondary treatment options such as surgery or medications associated with greater risk of side effects (i.e., steroids or anti-convulsants). Side effects commonly experienced by most patients treated with these riskier medications include fluid retention, weight gain, head pain, gastric distress, and swelling. Improved compositions and methods for the treatment of neuropathy are thus needed.

### SUMMARY

In at least one aspect, the present disclosure provides a physiologically buffered human umbilical cord (hUC) extract composition comprising micronized particles of ECM-degrading protease-treated hUC tissue. The hUC tissue may comprise, consist of, or consist essentially of hUC membrane, hUC stroma, or a combination of hUC membrane and hUC stroma. The composition may further comprise one or more gel forming agents, crosslinkers, biological molecules, enzymes, and/or buffers. For example, the composition may comprise an in situ polymerizing gel forming agent. The in situ polymerizing gel forming agent may be present at about 0.1 to 8 mg/ml.

The composition may further comprise a crosslinker, such as genipin or transglutaminase, among other suitable crosslinkers/crosslinking agents. The gel forming agent, e.g., polymerizing gel forming agent, e.g., a thermally polymerizing gel forming agent, may be or comprise one or more of fibrin, collagen I, collagen II, collagen III, collagen IV, collagen V, collagen VIII, collagen X, collagen XI, collagen XXIV, or collagen XXVII. In some examples, the ECM-degrading protease-treated hUC tissue comprises hUC membrane and the thermally polymerizing gel forming agent is not fibrin. The composition may be a saline-based suspension buffered at about pH 7.2 to 7.4.

The composition may further comprise one or more biological molecules, such as hyaluronic acid, chondroitin sulfate, chitosan, PEG, collagen VI, collagen VII, collagen IX, collagen XII, collagen XIII, collagen XIV, collagen XV, collagen XVI, collagen XVII, collagen XVIII, collagen XIX, collagen XX, collagen XXI, collagen XXII, collagen XXIII, collagen XXVI and/or collagen XXVIII. In at least one example, the composition does not comprise chondroitin sulfate. The composition may comprise micronized particles. For example, a majority of the micronized particles may have a diameter of between about 140 nm and about 160 nm.

Also provided is a method of producing such compositions. For example, the method may produce a human umbilical cord (hUC) extract, the method comprising (a) providing hUC membrane and/or stroma; (b) mechanically bombarding said hUC membrane and/or stroma to produce micronized particles; and (c) treating with an ECM-degrading protease, one or more of (i) the composition of step (a) prior to mechanical bombardment; (ii) the composition of step (b) during mechanical bombardment; and (iii) the micronized particles of resulting from step (b).

The methods herein may further comprise inactivating the protease. After inactivation of the ECM-degrading protease, an in situ gel forming agent, e.g., polymerizing gel forming agent, may be added. The method may further comprise polymerizing the gel forming agent. Polymerizing may occur in the presence of a crosslinker, such as genipin or transglutaminase.

The gel forming agent may be or comprise one or more of fibrin, collagen I, collagen II, collagen III, collagen IV, collagen V, collagen VIII, collagen X, collagen XI, collagen XXIV, or collagen XXVII. The in situ gel forming agent, e.g., polymerizing gel forming agent, may be present at about 0.1 mg/ml to 8 mg/ml. About 0.5-1.0 cm² of hUC tissue comprising hUC membrane and/or stroma may be provided in step (a). The hUC membrane and/or stroma provided in step (a) may be dispersed in a saline-based suspension buffered at between about pH 6.0 and 8.0.

The mechanical bombardment may be performed for between 1 and about 5 cycles. Further, for example, the mechanical bombardment may be performed with about a 60 second duration per cycle, at speeds ranging from, e.g., about 3400 RPM to about 3700 RPM. The methods herein may further comprise centrifuging the micronized particles prior to ECM-degrading protease treatment, such as at a speed of about 5000×g.

The protease, e.g., ECM-degrading protease, may be a collagenase or matrix metalloproteinase (MMP), such as Collagenase I, Collagenase III, MMP-2, MMP-3, or MMP-7. One or more of hyaluronic acid, chondroitin sulfate, chitosan, PEG, collagen VI, collagen VII, collagen IX, collagen XII, collagen XIII, collagen XIV, collagen XV, collagen XVI, collagen XVII, collagen XVIII, collagen XIX, collagen XX, collagen XXI, collagen XXII, collagen XXIII, collagen XXVI and/or collagen XXVIII may be added to the composition, e.g., after inactivation of the ECM-degrading protease. The mechanical bombardment may provide a majority of micronized particles having a diameter of between about 140 nm and about 160 nm, e.g., the particle size distribution having an average diameter ranging from about 140 nm to about 150 nm.

The present disclosure also includes methods of treating peripheral neuropathy comprising administering, e.g., injecting, a composition as described herein into a subject at or proximate a site of peripheral neuropathy. For example, the method of treating peripheral neuropathy may comprise injecting a composition made by a process as defined herein into a subject at or proximate a site of peripheral neuropathy. The subject may be a human, a primate, a non-human mammal, or another vertebrate or animal. The method may further comprise treating said subject with a second therapy such as analgesic therapy, NSAID treatment, and/or anti-convulsant medication. The methods herein may further comprise administering, e.g., injecting, said composition into said subject at least a second, third, fourth or fifth time, or on an ongoing or permanent, chronic, basis.

As used herein the specification, “a” and “an” mean one or more. As used herein, when used in conjunction with the word “comprising,” the words “a” and “an” mean one or more than one.

The use of the term “or” is used to mean “and/or” unless explicitly indicated to refer to alternatives only or the alternatives are mutually exclusive, although the disclosure supports a definition that refers to only alternatives and “and/or.” As used herein “another” means at least a second or two or more.

Throughout this application, the term “about” is used to indicate that a value includes the inherent variation of error for the device, the method being employed to determine the value, or the variation that exists among the study subjects. Unless noted otherwise, the term “about” should be understood to encompass ±5% of a specified amount or value.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present application and are included to further demonstrate certain aspects of the present disclosure. The disclosure may be better understood by reference to one or more of these drawings in combination with the detailed description of exemplary embodiments presented herein.

The patent or application file contains at least one drawing executed in color. Copies of this patent or patent application publication with color drawing(s) will be provided by the Office upon request and payment of the necessary fee.

FIGS. 1A-1B show preparation of hUC membrane extract as discussed in Example 1. (FIG. 1A) Pre-treatment hUC in tubes loaded at about 0.7 cm² of debrided hUC with 700 μL 1× PBS. (FIG. 1B) homogenized hUC when homogenized for 3 cycles at 6 m/s speeding setting for 60 sec in CoolPrep® sample holder followed by centrifuging at 5000×G for 10 minutes.

FIG. 2 shows a hUC membrane extract expelled from a syringe with a 26 G needle as discussed in Example 2.

FIG. 3 shows a collagen gel and hUC membrane extract polymerization as discussed in Example 2, including a 30 minute incubation at 37° C. in circular molds.

FIG. 4 shows a collagen gel and hUC membrane extract polymerization as discussed in Example 2. A pre-polymerized gel extract mixture was expelled from a syringe with a 26 G needle followed by 60 minutes incubation at 37° C.

FIGS. 5-6 show polymerization profiles as discussed in Example 3. hUC extract-loaded gels polymerized at 37° C. within 10 minutes without the addition of a gel crosslinker and within 8 minutes with a crosslinker added, indicating fast-acting in situ gel polymerization.

FIG. 7 shows gel degradation profiles as discussed in Example 2. Polymerized gels retained over 50% of their mass for a period of 1 day in physiological conditions without the addition of a crosslinker and for up to 36 days with the addition of a gel crosslinker.

FIGS. 8A-8B show particle size distributions as discussed in Example 3. (FIG. 8A) hUC extracts contained a monodisperse particle suspension with a predominant fraction of particles ranging between 160 and 180 nm in diameter. (FIG. 8B) Extracts contained a variable particle dispersity and size distribution profile depending on the preparation conditions.

FIG. 9 shows results of an immune modulation assay as discussed in Example 4. hUC extracts were observed to modulate the immune response of human peripheral blood mononuclear cells in vitro during an inflammatory challenge by changing their secretion response of immune biomarkers: IL-1b, IL-10, and MMP-9.

FIGS. 10A-10B show immune response modulation in human U-937 cell-line derived macrophage-like cells as discussed in Example 4. hUC extracts modulated the immune response of human U937 cell-line derived macrophage-like cells in vitro during an inflammatory challenge by altering their secretion response of immune biomarkers: IL-1β and IL-10.

FIG. 11 shows collagenase disruption of collagen polymer formation as discussed in Example 5. The concentration of disrupted collagen polymer fragments was decreased by treatment with collagenase.

FIG. 12 shows an exemplary schematic for preparing a hUC gel composition. hUC compositions may be prepared by mechanical processing of hUC tissue to obtain an extract, purifying the extract (e.g., removing cellular debris), treating the purified extract with a protease to reduce inflammatory components, and formulating the treated/purified hUC extract as a hydrogel suitable for injection.

FIG. 13 shows reduced expression of decorin as discussed in Example 6. hUC compositions treated with MMP-7 exhibited reduced expression of decorin (DCN) as compared to an untreated control.

FIG. 14A shows immune response modulation in human U-937 cell-line derived macrophage-like cells as discussed in Example 7. FIG. 14B reports total protein content for treated and untreated hUC extracts, as discussed in Example 7.

FIG. 15 shows an exemplary schematic for performing an immunomodulation assay, as discussed in Example 7.

FIGS. 16A-16B show results of an immunomodulation assay of protease-treated hUC extracts, as discussed in Example 7.

FIG. 17 shows reduced expression of decorin in protease-treated hUC extracts, as discussed in Example 7

FIGS. 18A-18B show results of another immunomodulation assay of protease-treated hUC extracts, as discussed in Example 7.

### DETAILED DESCRIPTION

As discussed above, the use of amniotic/birth materials is a promising avenue for treatment of tissue regeneration, including in the area of neuropathy. The present disclosure provides methods for generating improved biomaterials and methods of use therefore. In general, the disclosure is directed to a hUC material-derived saline-based suspension created by mechanical bombardment (e.g., bead-beating homogenization) of human umbilical cord (hUC) membrane and hUC stroma sections. This fabrication technique is designed to release a rich volume of soluble bioactive components relevant to inflammation modulation and restoration of healthy tissue into the saline-based suspension while reducing the soluble content of inflammatory components of the hUC membrane and stroma such as DNA, cytosolic DAMPs (damage associated molecular patterns), and ECM protein fragments in the same suspension. This suspension may be processed further to reduce inflammatory protein fragments by incubation with ECM-protease enzymes (e.g., collagenase or matrix metalloproteinase (MMP) such as MMP-7). This suspension may also be supplemented with collagen gel at a range of concentrations to enhance sustained delivery of therapeutic agents to the local tissue. This hUC membrane- and stroma-derived saline-based suspension may be delivered to a site of tissue injury (e.g., neuropathy) such as by injection. These and other features of the disclosure are described in detail below.

### I. NEUROPATHY

Neuropathy refers generally to nerve damage. Peripheral neuropathy describes damage to nerves other than those of the central nervous system, that is, damage to nerves other than those in the brain and spinal cord. For example, peripheral neuropathy encompasses damage to sensory and motor nerves connecting the brain and spinal cord to the rest of the body. (Damage to peripheral nerves can impair sensation, movement, and functionality, depending on the extent of damage and the peripheral nerves affected. Peripheral neuropathy encompasses damage that is reversible or permanent, where effects can be acute with sudden onset, rapid progress or chronic with symptoms that begin subtly and progress over time. Causes of peripheral neuropathy can be genetic or idiopathic (no known cause) and may accompany other medical conditions or prescribed medications.

### II. UMBILICAL CORD MATERIAL AND METHODS OF EXTRACTING THE SAME

The compositions herein are derived from umbilical cord tissue, including the membrane and/or stroma. Umbilical cord extracts may have benefits in treating neuropathy over materials derived from other types of tissues. Without being bound by theory, it is believed that the compositions herein derived from umbilical cord tissue may lead to reduced immune response, e.g., because umbilical cord tissue is deficient in human leukocyte antigen (HLA). Further, umbilical cord tissues comprise higher levels of bioactive growth factors, stem cells, free floating proteins, and glucosamine that may be beneficial in promoting nerve repair.

The compositions described herein can be prepared from human umbilical cord materials as described herein. These materials can be obtained from any suitable source. For example, at least one of the components can be obtained from human tissues. The components can be also obtained from commercial sources. The components can be purified, substantially purified, partially purified, or non-purified.

Human umbilical cord tissue can be obtained, for example, from commercial sources or from hospitals or surgical/maternity centers. The tissue is typically obtained in either a fresh or frozen state. The tissue can be washed to remove excess storage buffer, blood, or contaminants. The excess liquid can be removed, for example, using a brief centrifugation step, or by other means. The tissue can be frozen, using, for example, liquid nitrogen or other cooling means, to facilitate the subsequent homogenization. The source of the umbilical cord tissue can be a human umbilical cord (hUC). Whole hUC material may be debrided to remove materials extraneous to the membrane and/or stroma, e.g., through the use of surgical cutting tools, manual operated cutting machine, or automated cutting machine.

The hUC may also be processed to produce an extract through the use of mechanical bombardment, such as “bead-beating” techniques. Such processing may remove cellular debris. Bead-beating is a laboratory-scale mechanical method for processing biological samples using, for example, glass, ceramic or steel beads, mixed with a sample suspended in aqueous media. The sample and bead mix is subjected to agitation by, for example, stirring or shaking. Beads collide with the tissue material, mechanically disrupting the tissue to release therapeutic bioactive molecules. It has the advantage over other mechanical processing methods of being able to generate a micronized extract with a unique distribution of bioactive molecules and small particles, process many samples at a time with no cross-contamination concerns, and does not release potentially harmful aerosols in the process.

In an example of the method, an amount of beads, e.g., an equal volume of beads as compared to the amount of tissue, is added to a tissue suspension in a container, and the sample is vigorously mixed on a laboratory vortex mixer. While processing times may be relatively slow, taking 3-10 times longer than that in specialty shaking machines, it effectively processes tissue and is inexpensive. Scale up procedures with larger volumes and faster processing times are contemplated.

Successful bead-beating may depend not only on design features of the shaking machine (which take into consideration shaking oscillations per minute, shaking throw or distance, shaking orientation and vial orientation), but also the selection of correct bead size, bead composition (glass, ceramic, steel) and bead load in the vial.

High energy bead-beating machines typically warm the sample due to frictional collisions of the beads during homogenization. Cooling of the sample during or after bead-beating may be necessary to prevent damage to heat-sensitive proteins such as enzymes. Sample warming can be controlled by bead-beating for short time intervals and/or with cooling on ice/dry ice between each interval, by processing samples in pre-chilled aluminum vial holders, by circulating gaseous coolant through the machine during bead-beating. In some examples herein, samples in the processing chamber are cooled with dry ice, e.g., using a device from MP Biomedicals.

A different bead-beater configuration, suitable for larger sample volumes, uses a rotating fluorocarbon rotor inside a 15-, 50- or 200-ml chamber to agitate the beads. In this configuration, the chamber can be surrounded by a static cooling jacket. Using this same rotor/chamber configuration, large commercial machines are available to process many liters of cell suspension. Currently, these machines are limited to processing monocellular organisms such as yeast, algae and bacteria.

This initial processing may produce micronized particles of hUC tissue. For example, the hUC extract may comprise particles have a monomodal or bimodal particle size distribution, depending on the processing conditions. In some examples herein, the composition (before and/or after treatment with a protease as discussed further below) may have an average diameter ranging from about 50 nm to about 500 nm, such as from about 70 nm to about 250 nm, from about 80 nm to about 180 nm, from about 120 nm to about 350 nm, from about 150 nm to about 300 nm, from about 165 nm to about 200 nm, from about 140 nm to about 160 nm, from about 200 nm to about 275 nm, from about 175 nm to about 325 nm, or from about 250 nm to about 450. In some examples, particles above a given threshold may be removed (e.g., removing large proteoglycans and/or large tissue fragments, etc. released during homogenization), resulting in a desired monomodal or bimodal particle size distribution. Particle size may be measured, for example, by nanoparticle tracking analysis (NTA) or Multi Angle Dynamics Light Scattering (MADLS).

The tissue optionally can be frozen prior to the bombardment process. The freezing step can occur by any suitable cooling process. For example, the tissue can be flash-frozen using liquid nitrogen. Alternatively, the material can be placed in an isopropanol/dry ice bath or can be flash-frozen in other coolants. Commercially available quick-freezing processes can be used. Additionally, the material can be placed in a freezer and allowed to equilibrate to the storage temperature more slowly, rather than being flash-frozen. The tissue can be stored at any desired temperature. For example, −20° C. or −80° C. or other temperatures can be used for storage. Disruption of the tissue while frozen, rather than prior to freezing, is one optional method for preparing the tissue.

hUC preparations can be in a liquid, suspension, or dry (including, but not limited to, lyophilized) forms. Antimicrobial agents such as antibiotics or anti-fungal agents may be added. The material can be packaged and stored, for example, at room temperature, or for example, at −20° C. or −80° C. prior to use.

In some embodiments, the hUC material used to prepare the compositions, e.g., gel compositions, herein is present as a dry formulation. A dry formulation can be stored in a smaller volume and may not require the same low temperature storage requirements to keep the formulation from degrading over time. A dry formulation can be stored and reconstituted prior to use. The dry formulation can be prepared, for example, by preparing the freeze-morselized hUC as described herein, then removing at least a portion of the water in the composition. Water can be removed from the preparation by any suitable means. An exemplary method of removing the water is by use of lyophilization using a commercially available lyophilizer or freeze-dryer. Suitable equipment can be found, for example, through Virtis, Gardiner, N.Y.; FTS Systems, Stone Ridge, N.Y.; and SpeedVac (Savant Instruments Inc., Farmingdale, N.Y.). In certain embodiments, the water content of the dry formulation will be less than about 20%, down to about 10%, down to about 5% or down to about 1% by weight of the formulation. In some embodiments, substantially all of the water is removed. The lyophilized composition can then be stored. The storage temperature can vary from less than about −196° C., −80° C., −50° C., or −20° C. to more than about 23° C. If desired, the composition can be characterized (weight, protein content, etc.) prior to storage.

The lyophilized composition can be reconstituted in a suitable solution or buffer prior to use. Exemplary solutions include but are not limited to phosphate buffered saline (PBS), Dulbecco's Modified Eagle's medium (DMEM), and balanced salt solution (BSS). The pH of the solution can be adjusted as needed. The concentration of the hUC can be varied as needed. In some examples herein a more concentrated hUC solution is useful, whereas in other examples, a solution with a low concentration of hUC is useful. Additional compounds can be added to the solution. Exemplary compounds that can be added to the reconstituted formulation include but are not limited to pH modifiers, buffers, collagen, hyaluronic acid (HA), antibiotics, surfactants, stabilizers, proteins, and the like (discussed further below).

### II. UMBILICAL CORD EXTRACT COMPOSITIONS

In accordance with the present disclosure, there are provided hUC compositions as described above. These compositions may be further treated or supplemented with other materials as described herein, including, but not limited to, one or more gel forming agents, crosslinkers, biological molecules, enzymes, and/or buffers. The compositions herein may be formulated for administration to a subject, such as in solution or gel form.

A. Gel-Forming Agent(s)

The compositions herein may include one or more gel-forming agents. Suitable gel forming agents may be thermally-polymerizable at temperatures found in the human body, around 37° C. (98-99° F.). Exemplary gel forming agents include, but are not limited to, Collagens I, II, III, IV, V, VIII, X, XI, XXIV, and XXVII; polyethylene glycol (PEG); poly(lactic co-glycolic acid) (PLGA); poly(ethylene glycol) diacrylate (PEGDA); gelatin methacryloyl (GelMA); and methacrylated hyaluronic acid (MeHA); as well as fibrin. Fibrin, also called Factor Ia, is a fibrous, non-globular protein involved in the clotting of blood.

The amount of gel-forming agent generally may range from about 0.1 g/mL to about 8 mg/mL, such as about 0.5 g/mL to about 5 mg/mL, about 1 mg/mL to about 4 mg/mL, or about 3.5 mg/mL to about 4.5 mg/mL.

Collagen

In at least one aspect, the composition comprising hUC extract may include one or more collagen types. Fibril-forming or network-forming collagens including type I, II, III, IV, V, VIII, X, XI, XXIV, or XXVII may be employed as in situ polymerizing gel-forming agents (discussed below). Other collagens may be included in the composition as well.

In another aspect, the composition comprising hUC extract may include one or more collagen types, none of which are fibril-forming or network-forming.

Collagen is the main structural protein in the extracellular matrix in the various connective tissues in the body. As the main component of connective tissue, it is the most abundant protein in mammals, making up from 25% to 35% of the whole-body protein content. Collagen is made of amino acids bound together to form a triple helix of elongated fibril known as a collagen helix. It is mostly found in fibrous tissues such as tendons, ligaments, and skin.

Any one or more of the following may be included in the composition of the hUC extract:
Fibrillar (Type I, II, III, V, XI, XXIV, XXVII)
Non-fibrillar
FACIT (Fibril Associated Collagens with Interrupted Triple Helices) (Type IX, XII, XIV, XVI, XIX, XX, XXI, XXII)
Network-forming collagens (Type VIII, IV, X)
Multiplexin (Multiple Triple Helix domains with Interruptions) (Type XV, XVIII)
MACIT (Membrane Associated Collagens with Interrupted Triple Helices) (Type XIII, XVII)
Transmembrane associated collagen (XXIII)
Other (Type VI, VII, XXVI, XXVIII)

The five most common types are Type I (skin, tendon, vasculature, organs, bone (main component of the organic part of bone), Type II (cartilage; main collagenous component of cartilage), Type III (reticulate; main component of reticular fibers; commonly found alongside Type I), Type IV (forms basal lamina, the epithelium-secreted layer of the basement membrane) and Type V (cell surfaces, hair, and placenta).

Throughout the four phases of wound healing, collagen is understood to perform some or all of the following functions in wound healing:
Guiding function: Collagen fibers serve to guide fibroblasts. Fibroblasts migrate along a connective tissue matrix.
Chemotactic properties: The large surface area available on collagen fibers can attract fibrogenic cells, which help in healing.
Nucleation: Collagen, in the presence of certain neutral salt molecules, can act as a nucleating agent causing formation of fibrillar structures. A collagen wound dressing might serve as a guide for orienting new collagen deposition and capillary growth.
Hemostatic properties: Blood platelets interact with the collagen to make a hemostatic plug.

B. Crosslinkers

The compositions herein additionally or alternatively may comprise a crosslinker, also referred to herein as a crosslinking agent. Crosslinkers generally provide a bond that links one polymer chain to another. These links may take the form of covalent bonds or ionic bonds and the polymers can be either synthetic polymers or natural polymers (such as proteins). In polymer chemistry, “cross-linking” usually refers to the use of cross-links to promote a change in the polymers' physical properties. When “crosslinking” is used in biology, it generally refers to the use of an agent to link proteins together to check for protein—protein interactions or to create a strengthening of the overall biological material.

Exemplary crosslinkers useful for the present disclosure include, but are not limited to, genipin, transglutaminases, the imidoester crosslinker dimethyl suberimidate, the N-hydroxysuccinimide-ester crosslinker BS3, and formaldehyde. Further, for example, the compositions herein may comprise one or more photo-crosslinkable components, e.g., wherein UV light may be used to initiate crosslinking.

The crosslinkers dimethyl suberimidate, the N-hydroxysuccinimide-ester crosslinker BS3, and formaldehyde generally form a bond by inducing nucleophilic attack of the amino group of lysine and subsequent covalent bonding via the crosslinker. The zero-length carbodiimide crosslinker EDC functions by converting carboxyls into amine-reactive isourea intermediates that bind to lysine residues or other available primary amines. SMCC or its water-soluble analog, Sulfo-SMCC, may be used to prepare antibody-hapten conjugates for antibody development.

Genipin is a chemical compound found in gardenia fruit extract. It is an aglycone derived from an iridoid glycoside called geniposide present in fruit of Gardenia jasminoides. Genipin is a natural cross-linker for proteins, collagen, gelatin, and chitosan cross-linking. It has a low acute toxicity, with LD₅₀ i.v. 382 mg/kg in mice, therefore, much less toxic than glutaraldehyde and many other commonly used synthetic cross-linking reagents. Furthermore, genipin can be used as a regulating agent for drug delivery and as the intermediate for alkaloid syntheses. In vitro experiments have shown that genipin blocks the action of the enzyme uncoupling protein 2.

Another class of crosslinkers/crosslinking agents is the transglutaminases, which are enzymes that in nature primarily catalyze the formation of an isopeptide bond between γ-carboxamide groups (—(C═O)NH₂) of glutamine residue side chains and the ε-amino groups (—NH₃) of lysine residue side chains with subsequent release of ammonia (NH₃). Lysine and glutamine residues must be bound to a peptide or a protein so that this cross-linking (between separate molecules) or intramolecular (within the same molecule) reaction can happen. Bonds formed by transglutaminase exhibit high resistance to proteolytic degradation (proteolysis). These enzymes can also deamidate glutamine residues to glutamic acid residues in the presence of water. Transglutaminase isolated from Streptomyces mobaraensis bacteria for example, is a calcium-independent enzyme. Mammalian transglutaminases among other transglutaminases require Ca²⁺ ions as a cofactor.

Transglutaminases form extensively cross-linked, generally insoluble protein polymers. These biological polymers are indispensable for an organism to create barriers and stable structures. Examples are blood clots (coagulation factor XIII), as well as skin and hair. The catalytic reaction is generally viewed as being irreversible and must be closely monitored through control mechanisms.

The amount of crosslinker/crosslinking agent may range from about 0.1 mM to about 5 mM, such as about 0.5 mM to about 3 mM, about 3 mM to about 4 mM, about 1.5 mM to about 2.5 mM, or about 1 mM to about 2 mM.

C. Other Biological Molecules

In addition to the agents already discussed, the hUC compositions may further include one or more components such as, e.g., hyaluronic acid, chondroitin sulfate, chitosan, and/or polyethylene glycol (PEG).

Hyaluronic acid, also called hyaluronan, is an anionic, non-sulfated glycosaminoglycan distributed widely throughout connective, epithelial, and neural tissues. It is unique among glycosaminoglycans in that it is non-sulfated, forms in the plasma membrane instead of the Golgi apparatus, and can be very large: human synovial HA averages about 7 million Da per molecule, or about twenty thousand disaccharide monomers, while other sources mention 3-4 million Da. As one of the chief components of the extracellular matrix, hyaluronan contributes significantly to cell proliferation and migration, and may also be involved in the progression of some malignant tumors.

Chondroitin sulfate is a sulfated glycosaminoglycan (GAG) that includes a chain of alternating sugars (N-acetylgalactosamine and glucuronic acid). It is usually found attached to proteins as part of a proteoglycan. A chondroitin chain can have over 100 individual sugars, each of which can be sulfated in variable positions and quantities.

In some examples herein, the composition comprises a reduced amount of one or more types of proteoglycans (e.g., biglycan, decorin, versican, etc.) and/or sulfated GAGs associated with native hUC tissue. In some examples, the composition does not include one or more types of proteoglycans and/or sulfated GAGs associated with native hUC tissue. For example, the compositions herein may have a reduced amount of biglycan, decorin, and/or versican as compared to the native hUC tissue. In some examples, the compositions herein do not contain (e.g., have a level below detection) one or more proteoglycans present in the native hUC tissue, such as, e.g., biglycan, decorin, and/or versican.

Chitosan is a linear polysaccharide composed of randomly distributed β-(1→4)-linked D-glucosamine (deacetylated unit) and N-acetyl-D-glucosamine (acetylated unit) obtained from chitin. It is made by treating the chitin shells of shrimp and other crustaceans with an alkaline substance, like sodium hydroxide.

Polyethylene glycol (PEG) is a polyether compound also known as polyethylene oxide (PEO) or polyoxyethylene (POE), depending on its molecular weight. The structure of PEG is commonly expressed as H—(O—CH₂—CH₂)ₙ—OH. The possibility that PEG could be used to fuse axons is being explored by researchers studying peripheral nerve and spinal cord injury.

D. Enzymatic Processing

Following one or more processing steps (e.g., mechanical bombardment by bead-beating) of hUC tissue, the resulting hUC extract may comprise disrupted protein fragments such as protein monomers and ECM fragments. Such components may cause or lead to adverse effects when administered to a patient. For example, certain components of the hUC extract may provoke or be associated with an inflammatory response and/or immune response when injected at the site of nerve damage. The compositions herein may be prepared by treatment with a protease to selectively remove, reduce, or inactivate certain components while simultaneously retaining bioactive components useful in promoting nerve repair. Components that may be at least partially, substantially, or completely removed may include, for example, native proteoglycans such as decorin, biglycan, and/or versican.

Without being bound by theory, it is believed that the protease treatment may lead to degradation of or reduced expression of proteoglycans like decorin present in ECM associated with inflammation via the TLR4 pathway. For example, decorin is believed to affect inflammatory signaling events, being recognized by inflammatory cells as a DAMP. Removing or reducing the amount of proteoglycans like decorin may reduce the risk of inflammatory response by a subject when administered the compositions herein. Embodiments of the present disclosure may effectively guide the immune system to facilitate healing while reducing or preventing the potentially damaging aspects of the immune response.

For example, the making of the hUC compositions herein may include treatment with one or more ECM-degrading proteases. The protease may be a collagenase, such as Collagenase I, II, III, IV, V, VI or VII, or other suitable protease including, but not limited to, MMPs, such as MMP-2, MMP-3, or MMP-7. The enzyme treatment may precede the introduction of a gel-forming agent (discussed above) and/or may precede further introduction of one or more other components, including types of collagen and/or other gel forming agents as described above.

Collagenases are enzymes that break the peptide bonds in collagen. They assist in destroying extracellular structures in the pathogenesis of bacteria such as Clostridium. They are considered a virulence factor, facilitating the spread of gas gangrene. They normally target the connective tissue in muscle cells and other body organs. Collagen, a key component of the animal extracellular matrix, is made through cleavage of pro-collagen by collagenase once it has been secreted from the cell. This stops large structures from forming inside the cell itself. In addition to being produced by some bacteria, collagenase can be made by the body as part of its normal immune response. This production is induced by cytokines, which stimulate cells such as fibroblasts and osteoblasts, and can cause indirect tissue damage.

The concentration of protease may range from about 0.1 μg/mL to about 25 μg/mL, such as about 0.5 μg/mL to about 20 μg/mL, from about 1 μg/mL to about 15 μg/mL, about 0.5 μg/mL to about 5 μg/mL, about 1 μg/mL to about 2 μg/mL, about 2.5 μg/mL to about 5 μg/mL, about 3 μg/mL to about 8 μg/mL, about 5 μg/mL to about 15 μg/mL, about 10 μg/mL to about 18 μg/mL, about 15 μg/mL to about 22 μg/mL. Further, for example, the hUC extract may be treated with the protease for a period of time ranging from about 5 minutes to about 18 hours, such as about 1 hour to about 16 hours, about 4 hours to about 12 hours, about 8 hours to about 16 hours, about 12 hours to about 16 hours, about 2 hours to about 8 hours, about 30 minutes to about 5 hours, or about 5 minutes to about 2 hours. In a non-limiting example, the hUC extract may be treated with about 0.5 μg/mL to about 5 μg/mL of protease for a period of time ranging from about 1 hour to about 16 hours.

According to some examples herein, the method of preparing the composition includes at least partially inactivating the protease. For example, an agent such as, e.g., ethylenediaminetetraacetic acid (EDTA), ilomastat (e.g., GM-6001 or Galardin®), or TIMP metallopeptidase inhibitor 1 (TIMP-1) may be added to passivate the enzyme. Such agents may be selected to target the enzyme without damaging or minimizing damage to bioactive components in the hUC extract.

E. Buffers

The compositions, e.g., modified extracts, herein may be advantageously combined with a buffer solution to maintain a target pH. Typically, a buffer solution (e.g., pH buffer or hydrogen ion buffer) is an aqueous solution of a mixture of a weak acid and its conjugate base, or vice versa. Its pH changes very little when a small amount of strong acid or base is added to it. Buffer solutions are used to keep pH at a nearly constant value in a wide variety of chemical applications.

The pH of a solution containing a buffering agent typically varies within a limited range, regardless of what else may be present in the solution. In biological systems, this allows enzymes to serve their intended functions. If the pH value of a solution rises or falls too much, the effectiveness of an enzyme decreases in a process, known as denaturation, which may be irreversible. The majority of biological samples that are used in research are kept in a buffer solution, often phosphate buffered saline (PBS), at about pH 7.4.

Some exemplary buffering agents relevant to physiologic pH include citric acid and KH₂PO₄. By combining substances with pKₐ values differing by only two or less and adjusting the pH, a wide range of buffers can be obtained. Citric acid is a useful component of a buffer mixture because it has three pKₐ values, separated by less than two. The buffer range can be extended by adding other buffering agents. Various McIlvaine's buffer solutions, composed of Na₂HPO₄ and citric acid, have a buffer range of pH 3 to 8. Other buffers useful for biological systems suitable for the present disclosure include lactated Ringer's solution (LRS), tris(hydroxymethyl)aminomethane (TRIS), Hank's Balanced Salt Solution (HBSS), Gey's Balanced Salt Solution (GBSS), TAPSO, 4-(2-hydroxyethyl)-1-piperazineethanesulfonic acid (HEPES), N-[tris(hydroxymethyl)methyl]-2-aminoethanesulfonic acid (TES), 3-(N-morpholino)propanesulfonic acid (MOPS), piperazine-N,N′-bis(2-ethanesulfonic acid) (PIPES), cacodylate, and 2-(N-morpholino)ethanesulfonic acid (MES).

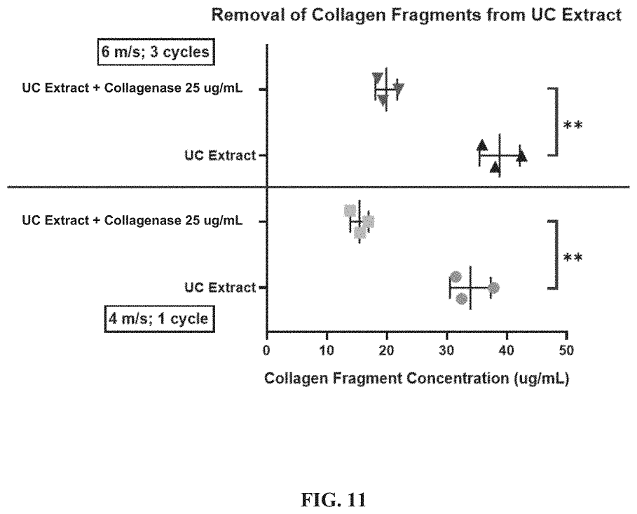

FIG. 12 shows an exemplary schematic for preparing a hUC gel composition in accordance with the discussion above and examples below. As shown, the compositions may be prepared by mechanical processing of hUC tissue (e.g., bead-beating or other suitable technique) to obtain an extract, purifying the extract (which may include, e.g., removing cellular debris), treating the purified extract with a protease to reduce inflammatory components, and formulating the treated/purified hUC extract as a hydrogel suitable for injection.

### IV. METHODS OF TREATMENT

In accordance with the disclosure, there are also provided methods for treating tissue injuries (including but not limited to, muscle, tendon, ligaments, etc.) in a subject, and in particular, treating neuropathy. The methods are designed to provide superior treatment of a site of pain, such as of peripheral neuropathy, compared with analgesic treatment or currently available amnion/birth-tissue-derived flowable products (e.g., OrthoFlo by Mimedx) and restore local tissues to a healthy state with normal function. This is intended to alleviate symptoms for patients who may undergo surgical intervention as well as patients who may not. As an injectable therapy, the methods are designed to be minimally invasive. The minimally invasive and versatile nature of this treatment as an injectable therapy intends to deliver accessibility to a treatment for a range of anatomical regions for painful neuropathy in the body.

Thus, in at least one aspect, the methods involve injection of hUC-derived material into a site of injury. Medical professionals are capable of assessing the appropriate site based on symptoms and diagnosis, which may include feet, hands, and joints, such as shoulders, elbows, wrists and knees. Similarly, doctors can determine the appropriate surgical procedures for delivery of the agents, such as combining injection with image guided delivery or surgical resection of affected regions.

As mentioned above, the compositions herein may be formulated as a gel, e.g., a hydrogel, for injection at or proximate to the site of injury or otherwise in need of treatment. Formulating the composition as a gel may provide for a longer duration of treatment, e.g., the gel may remain at the intended target site longer due to factors such as its viscosity, cohesion, etc. Accordingly, sustained release may be achieved by using gel compositions. The gel may be formulated to provide desired properties, such as degradation rate, density, stiffness, and/or cargo load.

The methods may include multiple treatments over a period of time, such as on an on-going or permanent, chronic basis. For example, any number of treatments, including 2, 3, 4, 5, 6, 7, 8, 9, 10, 15, 20, 25 or more treatments may be made. Such treatments may be spaced by days, weeks, months, or even years.

The methods may also include combination therapies employing administration of the hUC compositions described herein in conjunction with one or more recognized therapies for neuropathy, such as changes in diet and/or administration of NSAIDs, analgesics or anti-convulsants (discussed in greater detail above and incorporated herein by reference).

### V. EXAMPLES

The following examples are included to demonstrate exemplary embodiments of the disclosure without, however, being limiting in nature. It is understood that the present disclosure encompasses additional embodiments consistent with the foregoing description and following examples. It should be appreciated by those of skill in the art that the techniques disclosed in the examples that follow represent techniques discovered by the inventors to function well in the practice of the disclosure, and thus can be considered to constitute exemplary modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Preparation of hUC Extracts

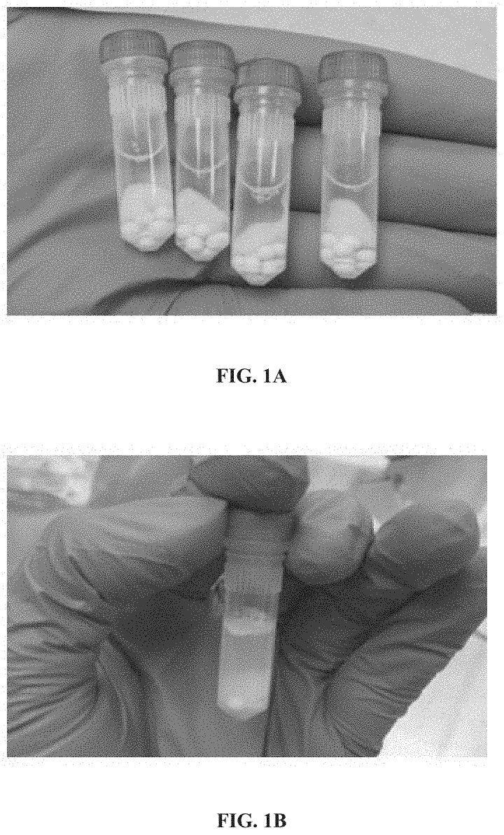

Compositions were prepared from hUC extracts as follows. Samples of hUC membrane tissue were loaded in tubes and 700 μL of 1× PBS was added (FIG. 1A). The samples were then homogenized for 3 cycles at a speed setting of 6 m/s for 60 seconds in a CoolPrep® sample holder followed by centrifuging at a speed of 5000×g for 10 min (FIG. 1B).

### Example 2

Preparation of hUC Gels

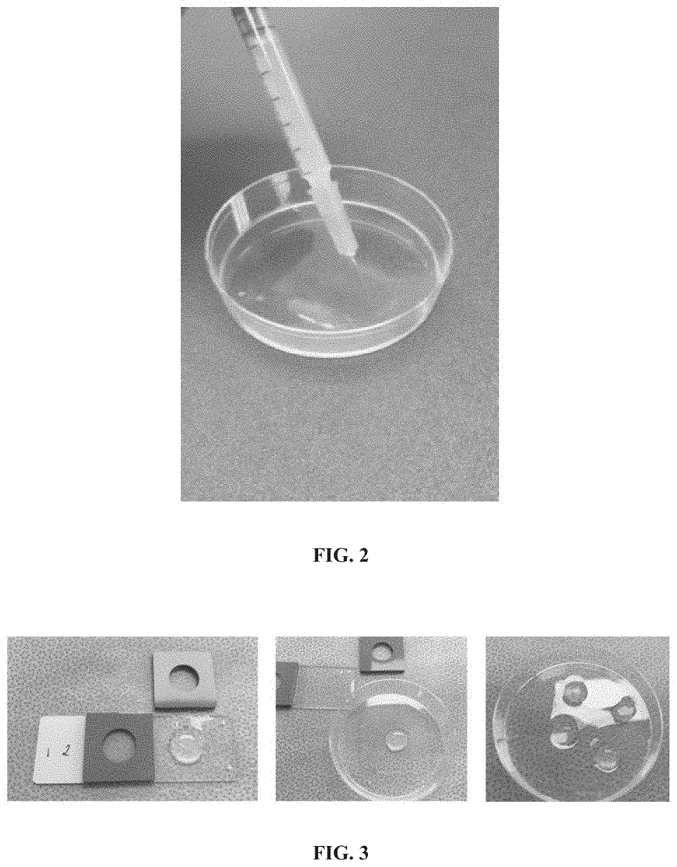

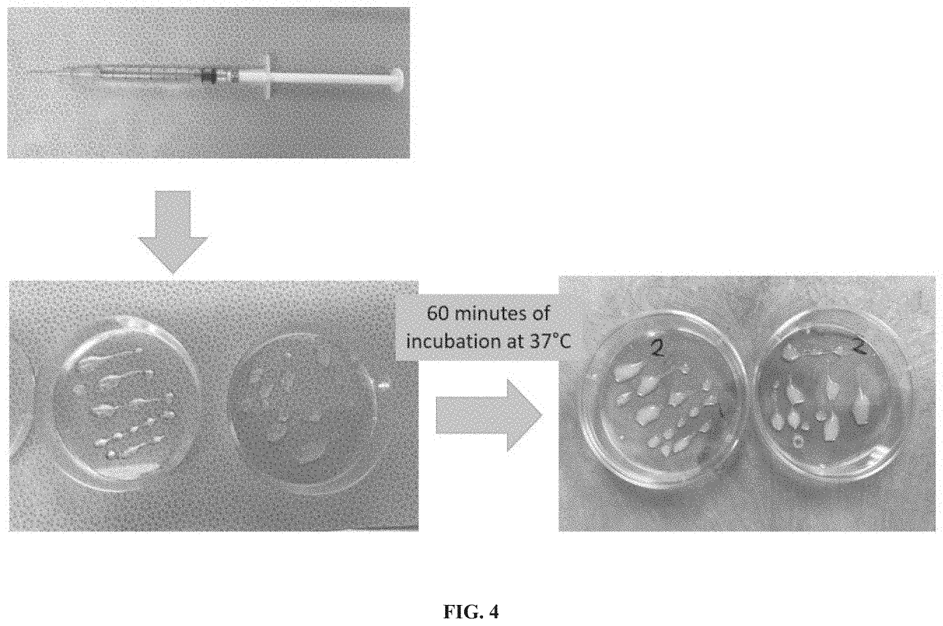

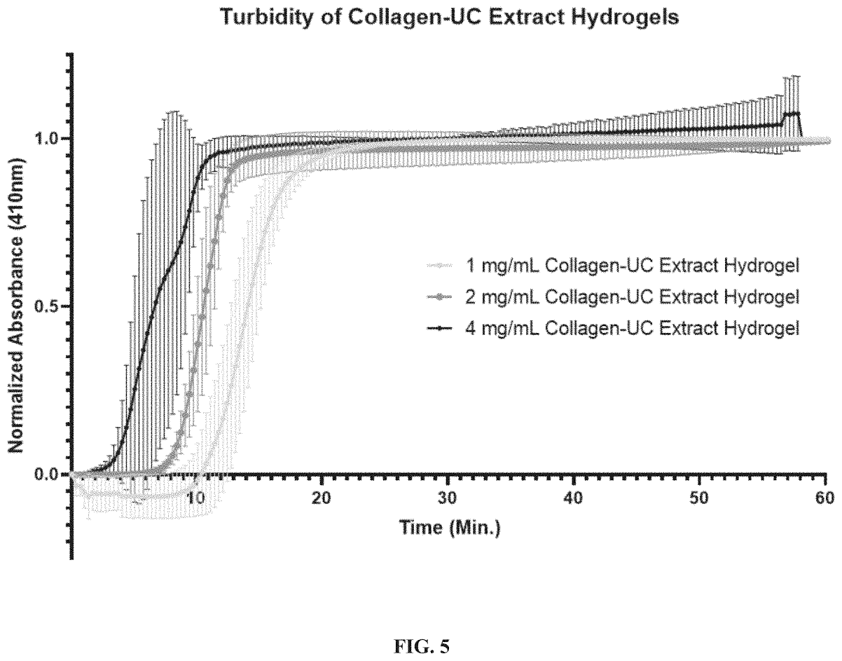

Studies were performed to assess formation of a gel with hUC extract and a gel forming agent. hUC extract prepared according to Example 1 was loaded into a syringe, extruded through a 26 G needle (FIG. 2), and polymerized into gel at 37° C. with collagen as a gel forming agent, e.g., a formulation suitable for use as a minimally invasive injectable therapy. FIG. 3 depicts collagen gel and hUC membrane extract polymerization after 30 minutes incubation at 37° C. in circular molds. FIG. 4 depicts collagen gel and hUC membrane extract polymerization, where a pre-polymerized gel extract mixture was expelled from a syringe with a 26 G needle followed by 60 minutes incubation at 37° C.

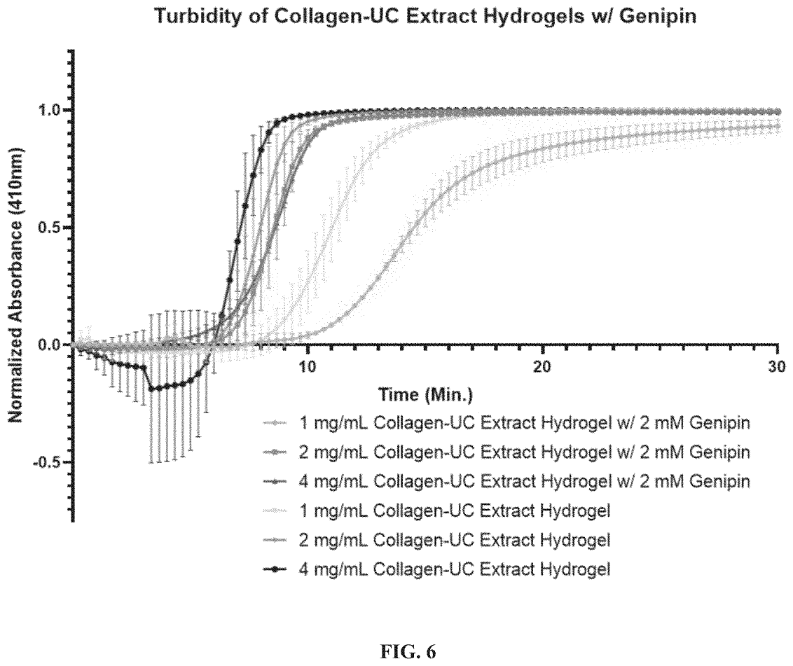

Samples were prepared with different amounts of collagen (1 mg/mL, 2 mg/mL, and 4 mg/mL) as a gel forming agent, and both with and without genipin (2 mM) as a crosslinking agent over a 30-minute incubation period to determine the effect on polymerization time. Absorbance at 410 nm was used as an indication of the degree of polymerization. Results are shown in FIGS. 5 and 6. hUC extract-loaded gels were observed to polymerize at 37° C. within 8 minutes without the addition of a gel crosslinker and within 10 minutes with a crosslinker added, indicating fast-acting in situ gel polymerization. Faster polymerization was observed with higher amounts of gel forming agent in the absence of a crosslinking agent. The sample prepared with 4 mg/mL collagen and without genipin exhibited the fastest polymerization time, followed by the sample prepared with 2 mg/mL collagen and without genipin. The samples prepared with 4 mg/mL and 2 mg/mL collagen, with 2 mM genipin exhibited similar polymerization times. The slowest polymerization was exhibited by the sample prepared with 1 mg/mL collagen and 2 mM genipin.

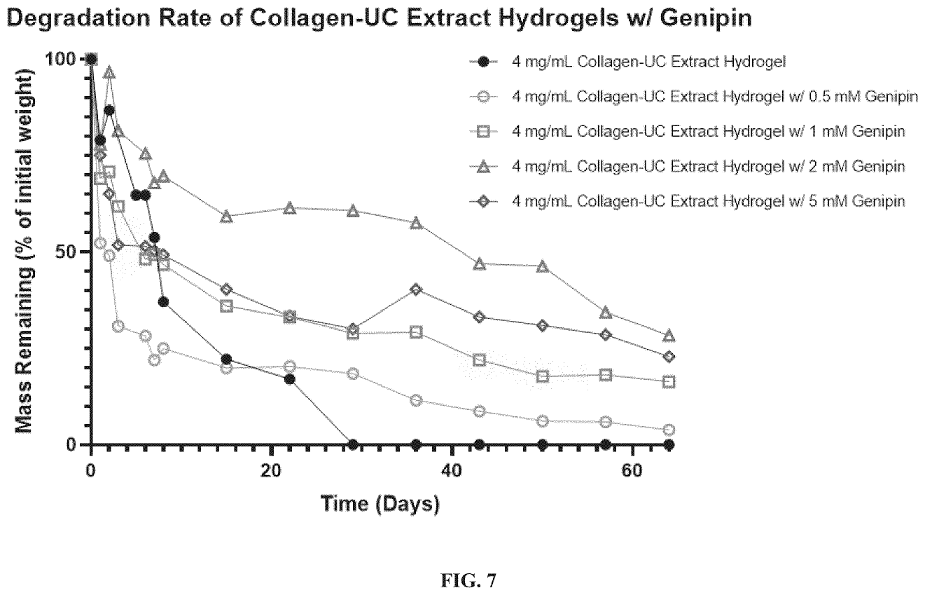

Further studies were done with samples prepared with hUC extracts combined with 4 mg/mL collagen as a gel forming agent and different amounts of genipin (0, 0.5 mM, 1 mM, 2 mM, and 5 mM) as a crosslinking agent, to investigate degradation of the polymerized gel over time (total 64 days). The samples were incubated in 1 mL of a digestive collagenase enzyme solution at 37° C. for 30 minutes over a period of 6 weeks. Studies were performed following polymerization and removal of excess liquid and then weighing weekly to determine starting mass and assess the ability to resist degradation. Fresh enzyme solution was used after each weighing. Polymerized gels were observed to retain over 50% of their mass for a period of 1 day in physiological conditions without the addition of a crosslinker, and for up to 36 days with the addition of a gel crosslinker (FIG. 7).

### Example 3

Characterization of hUC Extract Particle Size

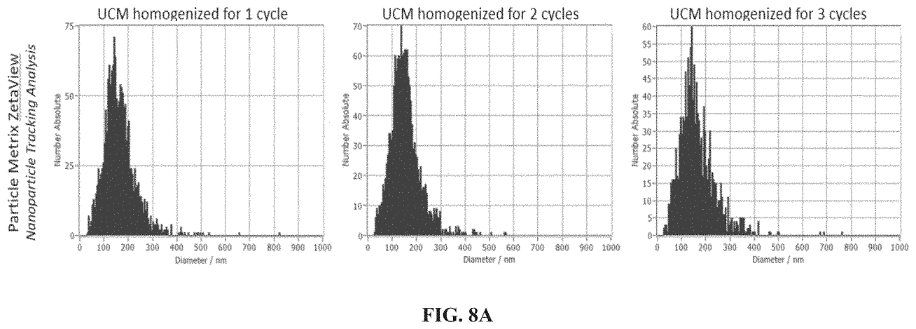

hUC extracts prepared according to Example 1 were analyzed to determine the particle size distribution. A Malvern Zetasizer Ultra analyzer was used to measure average diameter by Multi Angle Dynamics Light Scattering (MADLS). FIG. 8A shows results for samples in which the hUC membrane tissue (umbilical cord membrane (UCM)) was homogenized for 1 cycle, 2 cycles, and 3 cycles at 4 m/s at 60 seconds per cycle, showing a slightly broader size distribution for the longer homogenization time. These data support consistency in sample preparation, e.g., a consistent mixture of components from the homogenized hUC membrane tissue. The samples contained a monodisperse particle suspension with a predominant fraction of particles ranging between 160 and 180 nm in diameter (FIG. 8A). Further studies were done varying both the speed (4 m/s, 5 m/s, or 6 m/s) and the number of cycles (1, 3, or 5 cycles, with 60 seconds per cycle) to investigate the effect on particle size. The extracts contained a variable particle disperity and size distribution profile depending on preparation conditions (FIG. 8B).

### Example 4

Immunomodulation by hUC Extract

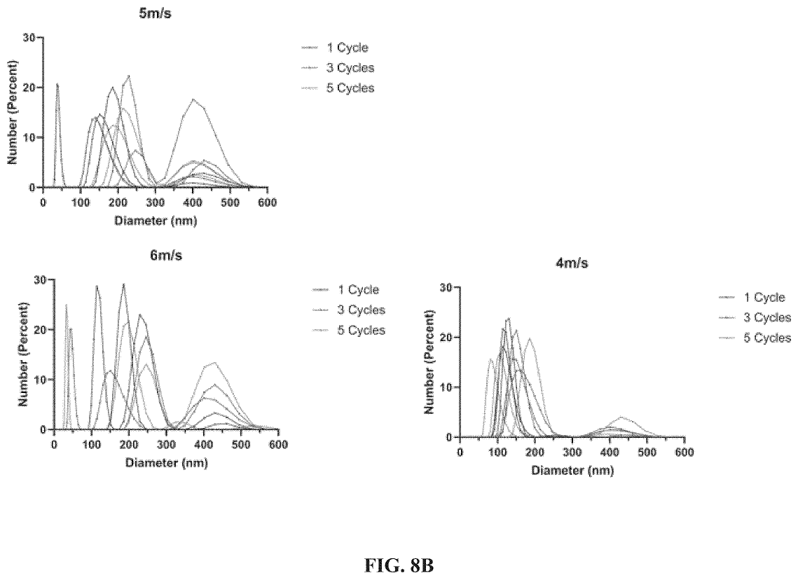

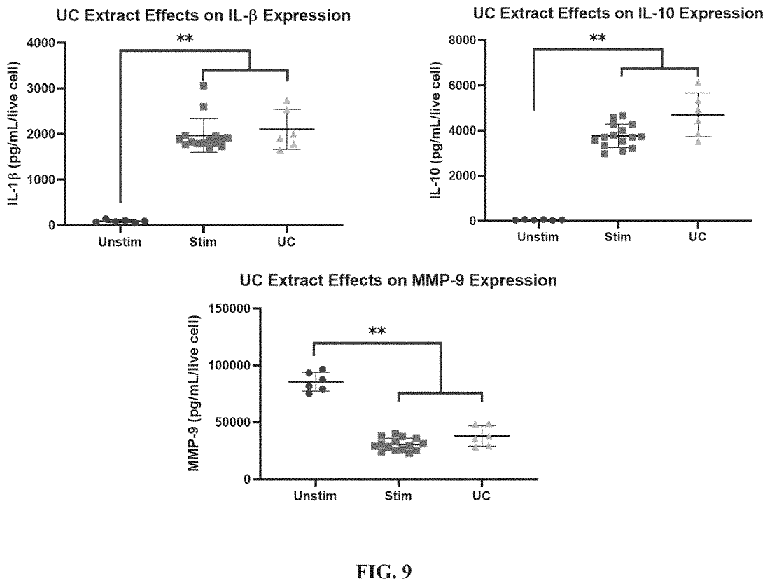

The ability for the hUC extracts to modulate the immune response of human peripheral blood mononuclear cells derived from the human U937 cell-line was investigated in vitro during two inflammatory challenges by changing their secretion response of the following immune biomarkers: IL-1β, IL-10, and MMP-9 (FIGS. 9 and 10A-10B). In the first study (FIG. 9), hUC extracts were prepared with 3 minutes homogenization of hUC membrane tissue per cycle at 4000 rpm, for a total of 4-5 cycles. In the second study (FIGS. 10A-10B), hUC extracts were prepared with 60 seconds homogenization of hUC membrane tissue per cycle at 6 m/s, for a total of 3 cycles. For the immunomodulation assays, U937 cell culture was treated with 20 ng/mL phorbol 12-myristate 13-acetate (PMA) for 48 hours to generate macrophage-like cells, then given M1 differentiation stimulus (50 ng/mL LPS+10 ng/mL IFN-γ) (“Stim”) or no stimulus (“Unstim”). M1 cultures were treated with the respective hUC extracts (“UC”) for comparison. Results show that the hUC extracts modulated the immune response of human U937 cell-line derived macrophage-like cells in vitro during an inflammatory challenge by altering their secretion response of immune biomarkers IL-1β and IL-10. FIG. 9 shows results at a 48-hour timepoint. FIGS. 10A-10B shows results at timepoints of 24, 48, 72, and 96 hours.

### Example 5

Protease (Collagenase) Treatment of hUC Extract

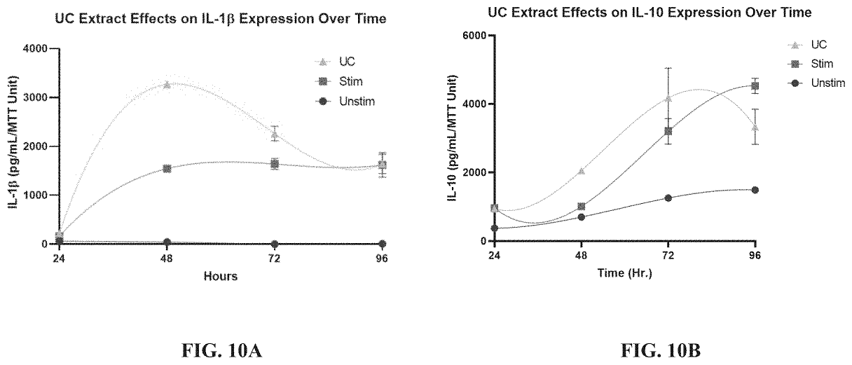

Studies were done with collagenase treatment (25 μg/mL) for comparison to a control with results shown in FIG. 11. As shown, the concentration of disrupted collagen polymer fragments decreased by treatment with collagenase as compared to the untreated control. The samples with collagenase treatment and the control samples were homogenized at speed settings of 4 m/s (1 cycle of 60 minutes) and 6 m/s (3 cycles of 60 minutes), and collagen polymer fragments decreased in both speed settings in the collagenase groups relative to the control groups.

### Example 6

Protease (MMP-7) Treatment of hUC Extract

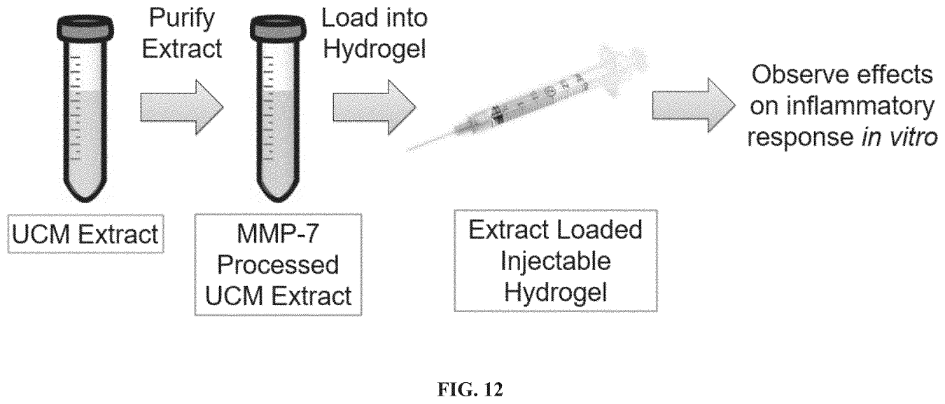

Modified/treated hUC extracts were prepared to study treatment by MMP-7 protease. hUC tissue was mechanically processed by 1 minute homogenization per cycle at 6 m/s, for a total of 3 cycles and then treated with MMP-7 (0.8 μg/mL) for 16 hours (“Treated UC”). This is shown schematically in the process of FIG. 12. A separate control was prepared without MMP-7 treatment (“UC”). As shown in FIG. 13, the extract treated with MMP-7 exhibited reduced expression of decorin (DCN) as compared to an untreated control. Decorin was measured with an ELISA kit (ThermoFisher Scientific).

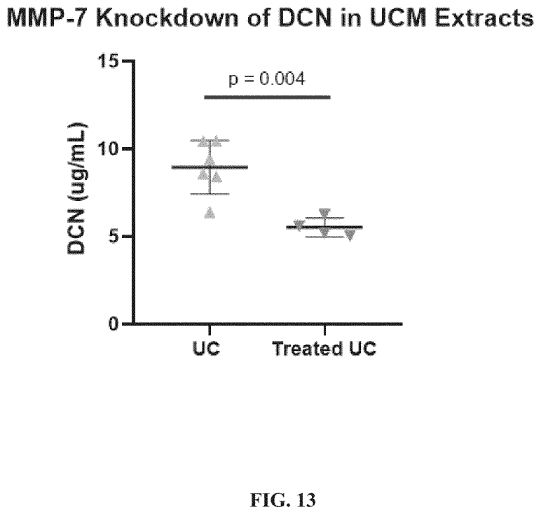

Additional hUC extracts were prepared and treated with MMP-7 under the same conditions as FIG. 13; decorin expression levels shown in FIG. 14A confirm reduction of about 45% decorin through the protease treatment. The difference in relative quantities of decorin may relate to variability among tissues, e.g., obtained from different donors or within the same tissue source. FIG. 14B shows the total protein content (μg/mL) measured for the treated and control samples, indicating similar levels. Total protein content was measured using a BCA (bicinchoninic acid) protein Assay Kit (ThrmoFisher Scientific) and tested 600 different proteins, of which 448 protein biomarkers were identified. This suggests that the MMP-7 treatment was successful in removing decorin content without significant damage to other proteins, including potentially beneficial components.

### Example 7

Immunomodulation by Protease-Treated hUC Extract

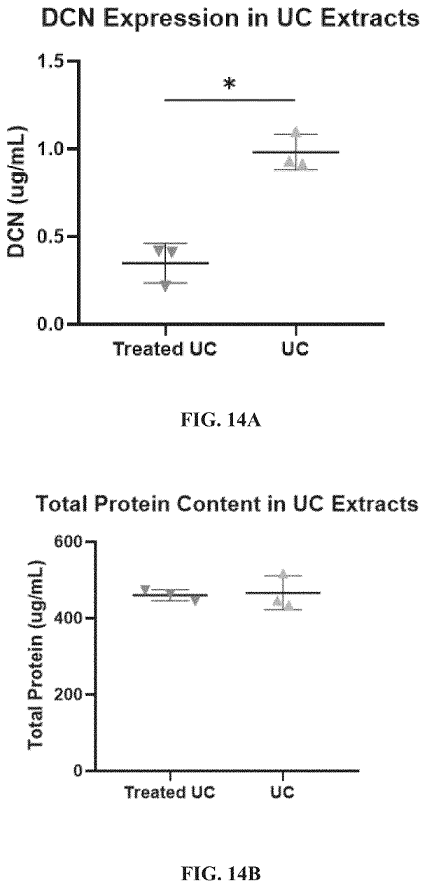

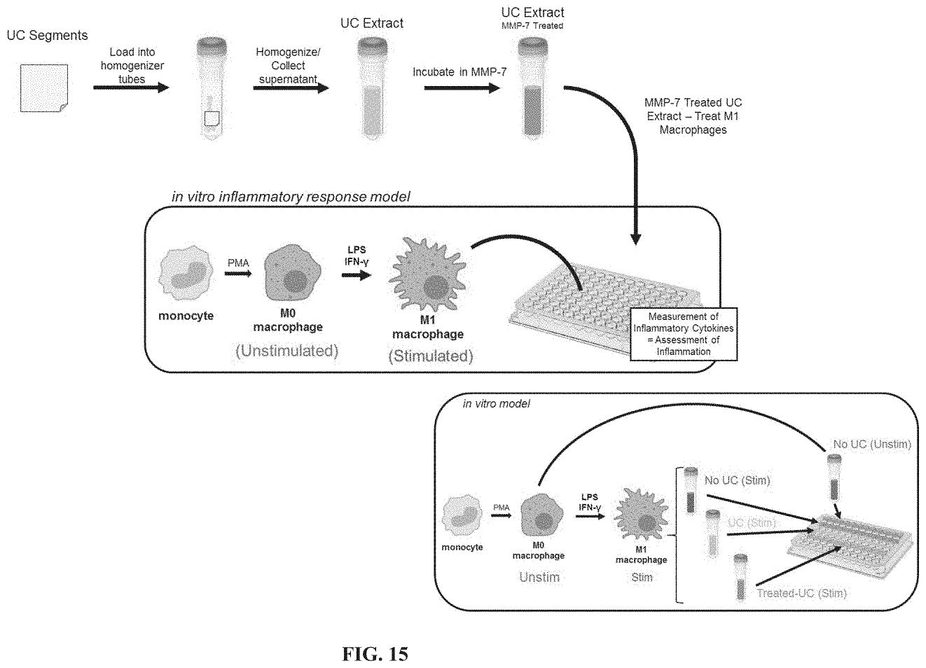

The samples of FIGS. 14A-14B prepared according to Example 6 were further investigated in an immunomodulation assay to determine their effect on immune biomarkers IL-1β and IL-10. U937 cell culture was treated with 20 ng/mL phorbol 12-myristate 13-acetate (PMA) for 48 hours to generate macrophage-like cells, then given M1 differentiation stimulus (50 ng/mL LPS+10 ng/mL IFN-γ) (“Stim”) or no stimulus (“Unstim”). M1 cultures were also treated with the untreated hUC extract (“UC”) and the MMP-7 treated hUC extract (“Treated UC”). The assay is illustrated schematically in FIG. 15. Results in FIGS. 16A and 16B shown that protease treatment led to reduced secretion response of IL-1β and IL-10. Without being bound by theory, it is believed that lower amounts of decorin in the treated hUC extracts led to reduced inflammatory response.

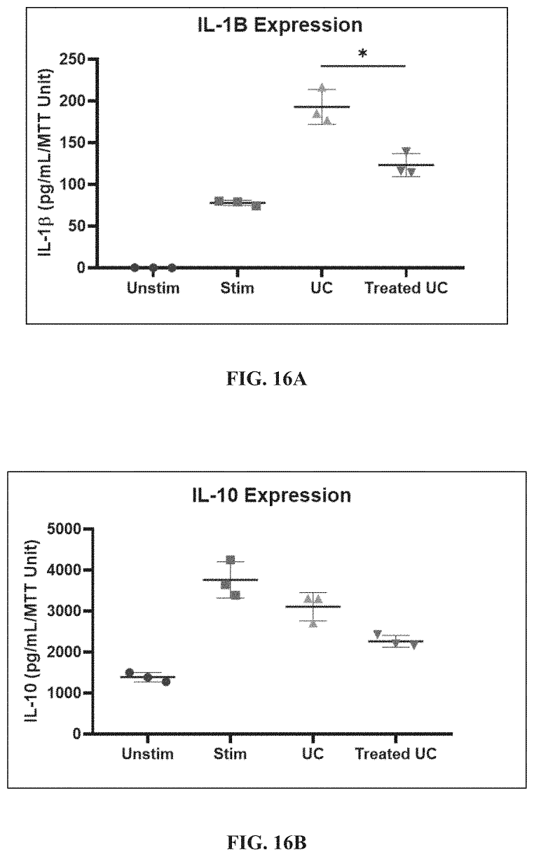

Additional studies were done to investigate an effect of residual protease from the treated hUC extracts on IL-1β and IL-10. hUC extracts were prepared and treated with MMP-7 as described in Example 6. Decorin levels of the untreated hUC extract control (“UC”) and the MMP-7 treated hUC extract (“Treated UC”) were measured as described in Example 6. The results shown in FIG. 17 confirm a reduction in decorin for the treated hUC extracts.

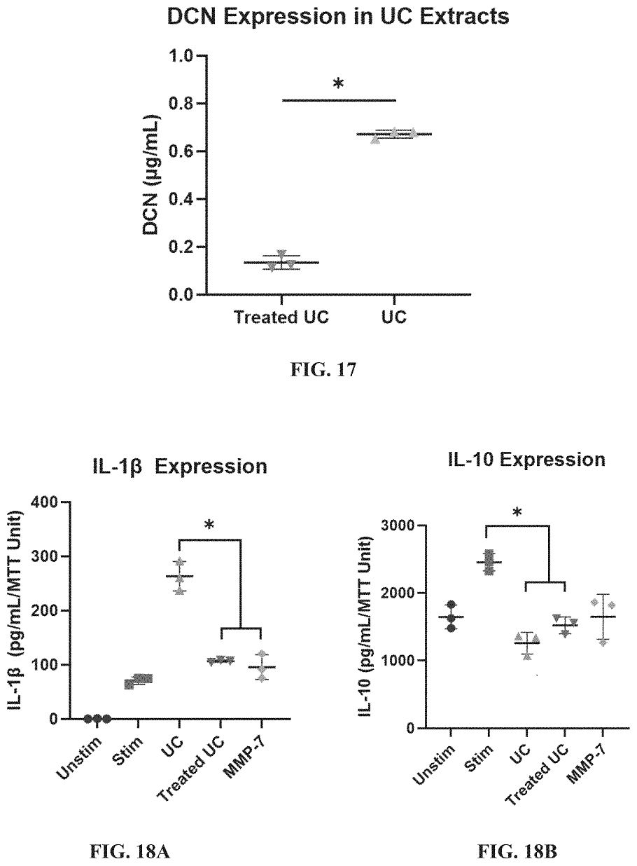

The treated and untreated hUC extract samples were subjected to an immunomodulation assay as described above. FIGS. 18A and 18B report the levels of IL-1β and IL-10, respectively, for unstimulated culture without hUC extract (“Unstim”), simulated culture without hUC extract (“Stim”), simulated culture with untreated hUC extract (“UC”), simulated culture with MMP-7 treated hUC extract (“Treated UC”), and stimulated culture with MMP-7 (0.8 μg/mL), without hUC extract (“MMP-7”). These results are consistent with those of FIGS. 16A-16B, showing that protease treatment of hUC extracts led to significant reductions in IL-1β and IL-10. MMP-7 alone was not observed to affect IL-1β as compared to the “Stim” control (FIG. 18A), and caused a slight (not statistically significant) reduction in IL-10 relative to control (FIG. 18B). This suggests that the reduction in inflammatory response is not due to residual protease, supporting the conclusion that removing decorin through protease treatment lowers immune response.

All of the methods disclosed and claimed herein can be made and executed without undue experimentation in light of the present disclosure and the knowledge of one of ordinary skill in this art. While the compositions and methods of this disclosure have been described in terms of exemplary embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed is:

1. A physiologically buffered human umbilical cord (hUC) extract composition comprising micronized particles of ECM-degrading protease-treated hUC.

2. The composition of claim 1, wherein the hUC comprises hUC membrane, hUC stroma, or a combination of hUC membrane and hUC stroma.

3. The composition of claim 1, further comprising a gel forming agent, e.g., an in situ polymerizing gel forming agent.

4. The composition of claim 3, wherein the gel forming agent is present at about 0.1 to 8 mg/ml.

5. The composition of claim 1, further comprising a crosslinker, such as genipin or transglutaminase.

6. The composition of claim 3, wherein the gel forming agent comprises one or more of fibrin, collagen I, collagen II, collagen III, collagen IV, collagen V, collagen VIII, collagen X, collagen XI, collagen XXIV, collagen XXVII, polyethylene glycol, poly(lactic co-glycolic acid, poly(ethylene glycol) diacrylate, gelatin methacryloyl, or methacrylated hyaluronic acid.

7. The composition of claim 1, wherein the ECM-degrading protease-treated hUC comprises hUC membrane, and the gel forming agent is not fibrin.

8. The composition of claim 1, wherein the composition is a saline-based suspension buffered at about pH 7.2 to 7.4, or wherein the composition is formulated as a gel such as a hydrogel.

9. The composition of claim 1, wherein the composition further comprises one or more of hyaluronic acid, chondroitin sulfate, chitosan, PEG, collagen VI, collagen VII, collagen IX, collagen XII, collagen XIII, collagen XIV, collagen XV, collagen XVI, collagen XVII, collagen XVIII, collagen XIX, collagen XX, collagen XXI, collagen XXII, collagen XXIII, collagen XXV, collagen XXVI and/or collagen XXVIII.

10. The composition of claim 1, wherein a majority of the micronized particles have a diameter of between about 80 nm and about 180 nm, such as between about 140 nm and about 160 nm.

11. A method of producing a human umbilical cord (hUC) extract comprising:
(a) providing hUC membrane and/or hUC stroma;
(b) mechanically bombarding said hUC membrane and/or hUC stroma to produce micronized particles; and
(c) treating with an ECM-degrading protease, one or more of (i) the composition of step (a) prior to mechanical bombardment; (ii) the composition of step (b) during mechanical bombardment; or (iii) the micronized particles of resulting from step (b).

12. The method of claim 11, further comprising inactivating the protease.

13. The method of claim 12, wherein after inactivation of the ECM-degrading protease gel forming agent, e.g., an in situ polymerizing gel forming agent, is added.

14. The method of claim 13, further comprising polymerizing the in situ gel forming agent.

15. The method of claim 14, where polymerizing occurs in the presence of a crosslinker.

16. The method of claim 15, wherein the crosslinker is genipin or transglutaminase.

17. The method of claim 13, wherein the gel forming agent comprises one or more of fibrin, collagen I, collagen II, collagen III, collagen IV, collagen V, collagen VIII, collagen X, collagen XI, collagen XXIV, collagen XXVII, polyethylene glycol, poly(lactic co-glycolic acid, poly(ethylene glycol) diacrylate, gelatin methacryloyl, or methacrylated hyaluronic acid.

18. The method of claim 13, wherein the gel forming agent is present at about 0.1 to 8 mg/ml.

19. The method of claim 11, wherein 0.5-1.0 cm² of hUC membrane and/or hUC stroma is provided in step (a).

20. The method of claim 11, wherein the hUC membrane and/or hUC stroma provided in step (a) is dispersed in a saline-based suspension buffered at between about pH 6.0 and 8.0.

21. The method of claim 11, wherein the mechanical bombardment is performed for between 1 and about 5 cycles, with about a 60 second duration per cycle, at speeds ranging from about 3400 RPM to about 3700 RPM.

22. The method of claim 21, further comprising centrifuging the micronized particles prior to ECM-degrading protease treatment.

23. The method of claim 11, wherein the ECM-degrading protease is a collagenase or matrix metalloproteinase (MMP).

24. The method of claim 23, wherein the protease is one or more of Collagenase I, Collagenase II, Collagenase III, Collagenase IV, Collagenase V, Collagenase VI, Collagenase VII, MMP-2, MMP-3, or MMP-7.

25. The method of claim 23, wherein the collagenase is one or more of Collagenase I or Collagenase III.

26. The method of claim 11, wherein one or more of hyaluronic acid, chondroitin sulfate, chitosan, PEG, collagen I, collagen II, collagen III, collagen IV, collagen V, collagen VI, collagen VII, collagen IX, collagen XII, collagen XIII, collagen XIV, collagen XV, collagen XVI, collagen XVII, collagen XVIII, collagen XIX, collagen XX, collagen XXI, collagen XXII, collagen XXIII, collagen XXVI or collagen XXVIII is added to the composition after inactivation of the ECM-degrading protease.

27. The method of claim 11, wherein the mechanical bombardment provides a majority of micronized particles having a diameter of between about 80 nm and about 180 nm, such as between about 140 nm and about 160 nm.

28. A method of treating peripheral neuropathy comprising injecting a composition according to claim 1 into a subject at a site of peripheral neuropathy.

29. A method of treating peripheral neuropathy comprising injecting a composition made by a process according to claim 11 into a subject at a site of peripheral neuropathy.

30. The method of claim 28, wherein the subject is a human.

31. The method of claim 28, further comprising treating said subject with a second therapy such as analgesic therapy, NSAID treatment, or anti-convulsant medication.

32. The method of claim 28, further comprising injecting said composition into said site a second time.

33. A composition comprising micronized particles of human umbilical cord (hUC) tissue and a buffer, wherein the composition is formulated for injection to a subject.

34. The composition of claim 33, wherein the composition is formulated as a gel.

35. The composition of claim 33, wherein the hUC tissue has been treated with a protease.

36. The composition of claim 33, wherein the composition comprises less than 1.0 μg/mL decorin.
